(19) United States
(12) Patent Application Publication
(10) Pub. No.: US 2026/0224460 A1
(43) Pub. Date: Aug. 6, 2026

(54) A PERSONAL CARE COMPOSITION

(71) Applicant: Conopco, Inc., d/b/a UNILEVER, Hoboken, NJ (US)

(72) Inventors: Chung-Ching CHU, Shanghai (CN); Vinitha KADAMKODE, Bangalore (IN); Rupak MITRA, Bangalore (IN); Barry Gerard MURPHY, Mancot (GB)

(73) Assignee: Conopco, Inc., d/b/a UNILEVER, Hoboken, NJ (US)

(21) Appl. No.: 19/154,022

(22) PCT Filed: Jan. 22, 2024
(86) PCT No.: PCT/EP2024/051358

§ 371 Date: Aug. 6, 2025

(30) Foreign Application Priority Data
Feb. 7, 2023 (WO) PCT/CN2023/074739
Mar. 14, 2023 (EP) 23161797.8

(51) Int. Cl. A61K 8/44; A61K 8/362; A61Q 19/08

(52) U.S. Cl. CPC A61K 8/44; A61K 8/362; A61Q 19/08

(57) ABSTRACT

The present invention relates to an antiaging composition. This is achieved through a topical composition comprising a specific amino acid and certain dicarboxylic acids or esters that delivers synergistic improvement in gene expression of Collagen1 which is an indicator of antiaging effect on skin.

### FIELD OF THE INVENTION

The present invention relates to an antiaging composition. This is achieved through a combination of a specific amino acid and certain dicarboxylic acids or esters thereof which when applied topically delivers this benefit.

### BACKGROUND OF THE INVENTION

Most people wish to have skin which appears youthful and resilient, and having uniform color distribution and smooth texture. However, human skin is subject to deterioration through, for example, dermatological conditions, exposure to environmental changes (wind, air conditioning, sun exposure, pollution, blue light etc.), or the normal aging with time (chrono aging). With time, individuals develop facial fine lines, wrinkles, yellowing or sallowness, sagging, hyperpigmentation, age spots and the general signs of aging. People resort to solutions to such problems through better nutrition, balanced exercise and a generally healthy lifestyle. While such activities are considered good for health and believed to slow the aging process, including delivering a visible appearance of younger looking skin, people prefer to use cosmetics, to at least temporarily, hide any signs of visible aging especially on the skin. Thus, the demand for anti-aging cosmetic products that address such visible signs has grown greatly.

Cosmetic compositions featuring retinoic acid precursors, such as retinol, have become quite prominent in recent years. Retinol, also known as vitamin A, and many of its ester derivatives and retinoid family members including, for instance, retinyl propionate, can be effective in reducing fine lines and wrinkles, smoothing skin, and improving uneven skin tone among other benefits, when applied topically. Notwithstanding the benefits of retinoids, the inclusion of retinoic acid precursors in cosmetic compositions often results in compositions having difficulties with stability. There is thus a need in the art to develop new combination of actives that deliver equivalent or even better anti-aging benefits. It is especially preferred that such actives are well known and proven for history of safe use on skin. The present inventors when seeking to develop such a technology hit up a combination of a very specific amino acid viz. arginine and certain dicarboxylic acids (viz. C7-C9 dicarboxylic acids) or esters thereof which work synergistically to deliver enhanced levels of collagen which is an indicator of antiaging benefits.

Arginine has been disclosed to have certain anti wrinkling benefit when used in a cream or ointment when used alone or in combination with certain other herbal ingredients e.g. in HU20040001422. The dicarboxylic acids or esters thereof as claimed in the composition of the present invention do not appear to have been known for this benefit. It was thus surprising for the present inventors when they found that a combination of these two class of compounds delivered synergistic improvement in gene expression of collagen1 which is an indicator of anti-aging benefit.

It is thus an object of the present invention to provide for a topical composition that delivers synergistic improvement in antiaging benefits to skin.

It is another object of the present invention to deliver this benefit through use of commonly used skin friendly compounds like amino acids and dicarboxylic acids.

### SUMMARY OF THE INVENTION

The first aspect of the present invention relates to a personal care composition comprising
(i) arginine;
(ii) a C₇ to C₉ dicarboxylic acid or ester thereof; and
(iii) a cosmetically acceptable vehicle;
wherein the weight ratio of arginine to the dicarboxylic acid or ester thereof is in the range of 50:1 to 1:1; and
wherein the composition does not comprise arginine and dimethyl pimelate in weight ratio 1:1.

Another aspect of the present invention relates to a method of delivering anti-aging benefit to skin comprising the step of applying a composition of the first aspect on to the desired skin surface.

### DETAILED DESCRIPTION OF THE INVENTION

These and other aspects, features and advantages will become apparent to those of ordinary skill in the art from a reading of the following detailed description and the appended claims. For the avoidance of doubt, any feature of one aspect of the present invention may be utilised in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated. Except in the operating and comparative examples, or where otherwise explicitly indicated, all numbers in this description and claims indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated.

The composition of the invention is meant to be used for personal care or for cosmetic use and could also be referred to as a personal care composition or a cosmetic composition. By a “personal care composition” as used herein, is meant to include a composition for topical application i.e external surfaces of the skin of humans. Such a composition may be classified as leave-on or rinse off, and includes any product applied to a human body for improving appearance, cleansing, odour control or general aesthetics. The composition is preferably of the leave-on type. The composition of the present invention can be in the form of a liquid, lotion, cream, foam, stick, serum, essence or gel. Preferred compositions include leave-on gels, lotions, serum or creams, preferably it is in the serum or cream form.

“Skin” as used herein is meant to include skin on the face and body (e.g., neck, chest, back, arms, hands, and legs) and especially to the exposed parts thereof.

The present invention relates to a personal care composition comprising (i) arginine and (ii) a C₇ to C₉ dicarboxylic acid or ester thereof; and (iii) a cosmetically acceptable vehicle.

Arginine is an amino acid with the chemical structure

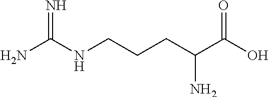 [SMILES: N=C(N)NCCCC(N)C(=O)O]

The molecule features a guanidino group appended to a standard amino acid framework. Arginine (Arg) residues are common components of proteins. Like all amino acids, it is a white, water-soluble solid. The present inventors have tried using other amino acids especially lysine which is of the similar class as arginine. They found that none of the other amino acids (including lysine) behave in a similar fashion as arginine in delivering synergistic anti-aging benefits in combination with the dicarboxylic acid or ester thereof as claimed in the present invention. Arginine may be included in 0.1 to 5%, preferably 0.5 to 2.5% by weight of the composition.

The composition of the invention comprises a C₇ to C₉ dicarboxylic acid or ester thereof. Preferred dicarboxylic acid or ester thereof are selected form one or more of pimelic acid, dimethyl pimelate, suberic acid and azelaic acid.

The chemical structures of the above compounds is as below:

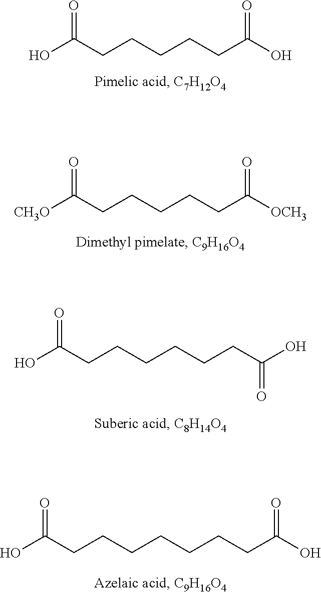

Most preferred dicarboxylic acid is pimelic acid. The dicarboxylic acid or ester thereof is preferably included in 0.05% to 1.5%, more preferably 0.1 to 1.0% by weight of the composition.

The weight ratio of arginine to the dicarboxylic acid or ester thereof is in the range of 50:1 to 1:1. Examples of such weight ratio include 50:1, 45:1, 40:1, 35:1, 30:1, 25:1, 5:1, 4:1, 3:1, 2:1 and 1:1. Accordingly, it is preferred that the weight ratio of arginine to the dicarboxylic acid or ester thereof, is preferably in the range of 45:1 to 1:1, preferably 40:1 to 1:1, preferably 35:1 to 1:1, preferably in the range 30:1 to 1:1, preferably 25:1 to 1:1, preferably 5:1 to 1:1, preferably 4:1 to 1:1, preferably 3:1 to 1:1, preferably 2:1 to 1:1.

For example, when the weight ratio of arginine to a dicarboxylic acid or ester thereof is 5:1, then, arginine is present in amounts that are 5 times more than the amount of a dicarboxylic acid or ester thereof. For example, when 0.005 wt % of arginine is taken, then the amount of pimelic acid is 0.001 wt %.

The composition does not comprise arginine and dimethyl pimelate in weight ratio 1:1.

Preferably, the composition does not comprise arginine and a C7 to C9 dicarboxylic acid or ester thereof in a weight ratio 20:1 or 10:1. Preferably, the composition does not comprise arginine and a C7 to C9 dicarboxylic acid or ester thereof in a weight ratio in the range of 20:1 to 10:1.

Preferably, the composition comprises arginine and a C7 to C9 dicarboxylic acid or ester thereof in such weight ratio that provides synergistic antiaging effect. Preferably antiaging effect is measured based on gene expression of collagen1 which is an indicator of anti-aging benefit.

The composition of the present invention may additionally comprise a known antiaging agent viz. a retinoid. Typically, the retinoid is selected from retinyl ester, retinol, retinal, retinoic acid or a mixture thereof. More preferably the retinoid comprises retinol, retinyl ester, or a mixture thereof. Particularly preferred retinoid is selected from all-trans-retinol, retinyl palmitate, retinyl acetate, retinyl propionate, or a mixture thereof. Most preferably the retinoid is selected from retinyl palmitate, retinyl propionate, or a mixture thereof.

Preferably, retinoid is employed in the composition in an amount of 0.001 to 0.5% and more preferably in an amount of 0.01% to 0.2% by weight of the composition.

The composition of the invention preferably includes a suitable fatty alcohol. Fatty alcohol for use in the present invention includes those compounds having from 10 to 20 carbon atoms in the fatty chain. It is especially preferred that the fatty alcohol is one of a cetyl, myristyl, palmityl or stearyl alcohol. The most preferred fatty alcohol for use in the present invention is cetyl alcohol. The fatty alcohol is preferably included in 0.1 to 2%, preferably 0.4 to 1.0% by weight of the composition.

The composition of the invention preferably includes a humectant. Humectants are compounds that help retain the loss of moisture from composition. The humectants are preferably selected from one or more of glycerol, sorbitol, saccharide isomerate, 1,2-hexane diol, 2,3-butanediol, panthenol, pentylene glycol, propanediol, polyglyceryl laurate, sodium PCA, lactic acid, sodium lactate, potassium lactate, acetamide MEA, hydroxyethyl urea, sodium hyaluronate, or hydrolyzed hyaluronic acid.

Preferably, the composition further comprises one or more skin glow agents. These agents may be selected from niacinamide, picolinamide, iso-nicotinamide, resorcinol, phenylethyl resorcinol, 4-alkyl resorcinol, vitamin B6, vitamin C, vitamin A, glutathione precursors, galardin, adapalene, ammonium lactate, arbutin, butyl hydroxy anisole, butyl hydroxy toluene, citrate esters, deoxyarbutin, 1,3-diphenyl propane derivatives, 2,5-dihydroxybenzoic acid and its derivatives, 2-(4-acetoxyphenyl)-1,3-dithiane, 2-(4-hydroxyphenyl)-1,3-dithiane, ellagic acid, gluco pyranosyl-1-ascorbate, gluconic acid, glycolic acid, 4-Hydroxy-5-methyl-3 [2H]-furanone, 4-hydroxyanisole and its derivatives, 4-hydroxybenzoic acid derivatives, hydroxycaprylic acid, inositol ascorbate, lactic acid, linoleic acid, magnesium ascorbyl phosphate, 5-octanoyl salicylic acid, salicylic acid, 3,4,5-trihydroxybenzyl derivatives, acetylglucosamine, symwhite, calcium pantothenate (Melano-block), seppiwhite, 12-hydroxystearic acid and mixtures thereof. More preferred skin glow agents for use in the composition include niacinamide, picolinamide, isonicotinamide, 12-hydroxystearic acid, resorcinol, phenylethyl resorcinol, 4-alkyl resorcinol, conjugated linoleic acid (CLA) or galardin. Even more preferred skin glow agents are selected from niacinamide, CLA and 4-alkyl resorcinol; and mixtures thereof. Examples of 4-alkyl substituted resorcinol include 4-methyl resorcinol, 4-ethyl resorcinol (ER), 4-propyl resorcinol, IPR, 4-butyl resorcinol (BR), 4-pentyl resorcinol, HR, 4-heptyl resorcinol, 4-octyl resorcinol and mixtures thereof. Preferred 4-alkyl substituted resorcinol are ER, BR, HR and mixtures thereof. More preferred 4-alkyl substituted resorcinol are ER, HR and mixtures thereof. The alkyl group in 4-alkyl substituted resorcinol can be straight chain alkyl or branched chain alkyl. For example, the alkyl group can be straight chain alkyl as in the case of 4-propyl resorcinol or it can be a branched chain alkyl like in the case of 4-isopropyl resorcinol (IPR). Preferably the composition comprises from 0.1 to 5 wt %, more preferably from 0.1 to 3 wt % and further more preferably from 0.1 to 1 wt % skin glow agent.

The composition may additionally comprise sunscreen agents such as inorganic sunblocks. For example, zinc oxide, titanium dioxide, iron oxide, and silica such as fumed silica. The amount of such sunblock agents is preferably incorporated from 0.1 to 5% by total weight of the sunscreen composition.

The composition of the invention may comprise an organic UV-A sunscreen agent selected from the group consisting of a dibenzoylmethane derivative, a triazine derivative, a benzophenone derivative and mixtures thereof. In a preferred embodiment, the UV-A sunscreen agent comprises or is a dibenzoylmethane derivative, for example, butyl methoxydibenzoylmethane (sold under the trade name Parsol 1789).

Typically, the composition of the present invention may comprise from 0.1 to 15% by weight of the UV-A sunscreen agent, more preferably from 0.1 to 10%, most preferably from 1 to 5%, based on the total weight of the composition and including all ranges subsumed therein.

The composition of the invention may also comprise an organic UV-B sunscreen agent. Suitable UV-B sunscreen agent may be selected from the group consisting of a benzophenone, an anthranilate, a salicylate, a cinnamate, a camphor, benzylidene malonate, a triazone, and derivatives thereof. In a preferred embodiment, the organic UV-B sunscreen agent comprises or is a cinnamate derivative, for example, ethylhexyl methoxycinnamate (sold under the trade name Parsol MCX).

Typically, the composition comprises from 0.1 to 20% by weight of the UV-B sunscreen agent, more preferably from 0.5 to 18%, most preferably from 1 to 15%, based on the total weight of the composition and including all ranges subsumed therein.

The composition of the invention comprises a cosmetically acceptable vehicle. The cosmetically acceptable vehicle is preferably in the form of an oil, liquid, stick, cream, lotion, spray or gel. The cosmetically acceptable vehicle preferably includes ingredients like surfactant e.g. non-ionic surfactant, fatty acid, soap, water, polymer, emollients, solvents, powders, preservatives, or optional ingredients. Details on the various possible ingredients for inclusion in the cosmetically acceptable vehicle are given below.

Preferably, the composition comprises a nonionic surfactant. More preferably the nonionic surfactant is selected from those having HLB value in the range 9 to 20, preferably 10 to 19, more preferably 12 to 18, even more preferably 13 to 17 and yet more preferably 15 to 17. HLB is calculated using the Griffin method wherein HLB=20×Mh/M wherein Mh is the molecular mass of the hydrophilic portion of the molecule and M is the molecular mass of the whole molecule, giving a result on an arbitrary scale of 0 to 20.

Preferably, the nonionic surfactant having HLB value in the range 9 to 20 is selected from fatty alcohol ethoxylates, alkyl phenol ethoxylates, polyoxyethylene sorbitan alkyl esters and mixtures thereof. Preferably, the nonionic surfactants are ones with at least 9 alkylene oxide groups preferably at least 9 ethylene oxide groups.

Examples of fatty alcohol ethoxylates that may be used as nonionic surfactants in the composition include polyoxyethylene lauryl ether (HLB=16.9; commercially available as Brij® 35), polyoxyethylene (20) cetyl ether (HLB=16; commercially available as Brij® 58), polyethylene glycol octadecyl ether (HLB=18.8; commercially available as Brij® 700) and Laureth-9 (C12EO9; HLB=14.3; commercially available as Brij® L9).

Examples of alkyl phenol ethoxylates that may be used as nonionic surfactant in the composition include octylphenol ethoxylate (HLB=15.5; commercially available as Triton™ X165), octylphenol ethoxylate (HLB=17.6; commercially available as Triton™ X405) and octylphenol ethoxylate (HLB=18.4; commercially available as Triton™ X705).

Examples of polyoxyethylene sorbitan alkyl esters that may be used as the nonionic surfactant in the composition include polyoxyethylenesorbitan monolaurate (HLB=13.3; commercially available as Tween® 21), polyoxyethylenesorbitan monolaurate (HLB=16.7; commercially available as Tween® 20), Polyoxyethylenesorbitan monopalmitate (HLB=15.6; commercially available as Tween® 40) and polyoxyethylene sorbitan monostearate (HLB=14.9; commercially available as Tween® 60).

Even more preferably, the nonionic surfactant having HLB value in the range 9 to 20 that may be present in the composition is fatty alcohol ethoxylate with saturated carbon chain having HLB higher than 15.5.

Preferably, the composition comprises 0.5 to 5 wt %, more preferably 1 to 4 wt %, even more preferably from 2 to 3 wt % nonionic surfactant having HLB in the range 9 to 20.

Preferably, the composition of the invention is delivered in the form of a vanishing cream. A vanishing cream is one which when applied and rubbed on to the human skin, vanishes on the skin leaving behind no significant streaks of the composition. Fatty acids when present in a composition along with a soap provides the so-called vanishing cream effect. Preferably, the leave-on composition comprises fatty acids having 10 to 30, more preferably 12 to 25, even more preferably 14 to 20, further more preferably 16 to 18 carbon atoms. Examples of fatty acids that may be used in the composition include pelargonic, lauric, myristic, palmitic, stearic, isostearic, oleic, linoleic, arachidic, behenic, erucic acid and mixtures thereof. Preferably, the fatty acid that may be used is stearic acid or palmitic acid or a mixture thereof. The fatty acid in the present invention is preferably hystric acid which is substantially (generally about 90 to 95%) a mixture of stearic acid and palmitic acid in a ratio of between 55:45 to 45:55.

Preferably, the composition comprises from 2 to 25 wt %, more preferably 6 to 20 wt %, further more preferably from 10 to 18 wt % fatty acid.

Preferably, the composition comprises soap. Soap, when present in combination with fatty acid in the composition, provides the vanishing effect described above. Preferably, soap in the composition is generally prepared by in-situ neutralization of fatty acid that may be present in the composition. Thus, it is preferred that the soap has a carbon chain length that corresponds to the chain length of fatty acid in the composition. The soap is formed from the fatty acid through use of alkali metal hydroxides e.g. sodium hydroxide or potassium hydroxide. Of the two, potassium hydroxide is more preferred. Thus, the soap is preferably a potassium soap (potassium salt of fatty acid).

Preferably, the composition comprises from 0.1 to 10%, more preferably from 0.5 to 7%, further more preferably 0.5% to 3% soap by weight of the composition.

Preferably, the composition comprises water in amount from 5 to 99.9 wt %, more preferably from 10 to 95 wt %, even more preferably from 15 to 90 wt %, further more preferably from 20 to 80 wt %, still more preferably 25 to 75 wt % and yet more preferably 30 to 70 wt %.

Preferably, the composition comprises a polymer. The polymer acts as thickener in the composition and improves sensorial properties of the composition.

The polymer is preferably selected from the following classes:
acrylate/R-methacrylate copolymer e.g. acrylates/steareth-20 methacrylate copolymer (commercially available as Aculyn™ 22) and acrylates/beheneth-25 methacrylate copolymer (commercially available as Aculyn™ 28),
acrylate/R-methacrylate crosspolymer e.g. acrylates/steareth-20 methacrylate crosspolymer (commercially available as Aculyn™ 88),
acrylates copolymer (commercially available as Aculyn™ 33),
acrylate/R-alkyl acrylate crosspolymer e.g. acrylates/C10-C30 alkyl acrylate crosspolymer (commercially available as Pemulen™ TR-2),
copolymer of ammonium acryloyldimethyltaurate with vinyl pyrrolidone (commercially available as Aristoflex® AVC),
copolymer of sodium acryloyldimethyltaurate with vinyl pyrrolidone (commercially available as Aristoflex® AVS); and
crosspolymer of acryloyldimethyltaurate with R-alkyl acrylate and methyacrylate e.g. Ammonium acryloyldimethyltaurate/beheneth-25 methacrylate crosspolymer (commercially available as Aristoflex® HMB and Aristoflex® BLV).

Preferably, the composition comprises 0.1 to 5 wt %, more preferably 0.5 to 4 wt %, still more preferably from 0.75 to 2.75 wt % polymer.

Preferably, the composition comprises emollients. Examples of emollients that may be used in the leave-on composition include stearyl alcohol, glyceryl monoricinoleate, mink oil, isopropyl isostearate, isobutyl palmitate, isocetyl stearate, oleyl alcohol, isopropyl laurate, hexyl laurate, decyl oleate, 10ummarized-2-ol, isocetyl alcohol, eicosanyl alcohol, behenyl alcohol, cetyl palmitate, silicone oils such as dimethylpolysiloxane, din-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rape seed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isopropyl linoleate, lauryl lactate, myristyl lactate, decyl oleate, myristyl myristate and mixtures thereof.

Preferably, the composition comprises solvents. Examples of solvents that may be used in the composition include ethyl alcohol, isopropanol, acetone, ethylene glycol ono ethyl ether, diethylene glycol mono butyl ether, diethylene glycol mono ethyl ether and mixtures thereof. Preferably, the composition comprises powders. Examples of powders that may be used in the composition include chalk, talc, fullers earth, kaolin, starch, gums, colloidal silica sodium polyacrylate, tetra alkyl and/or trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose, ethylene glycol monostearate and mixtures thereof.

Preferably, the composition comprises preservatives to protect against the growth of potentially harmful microorganisms. Examples of ingredients that may be used as preservatives in the composition include alkyl esters of para-hydroxybenzoic acid, hydantoin derivatives, propionate salts, and a variety of quaternary ammonium compounds. More preferably, ingredients that may be used as preservative in the composition are sodium benzoate, iodopropynyl butyl carbamate, methylisothiazolinone, iodopropynylbutylcarbamate, phenoxyethanol, methyl paraben, propyl paraben, imidazolidinyl urea, sodium dehydroacetate, ethylhexylglycerin, benzyl alcohol, alkane diols and mixtures thereof. The alkane diols that are suitable for use as preservative are C₆-C₁₂ alkanes that are vicinally substituted with hydroxy groups. Illustrative examples include 1,2-octane diol (caprylyl glycol), 2,3-octane diol, 1,2-nonane diol, 1,2-decane diol, 1,2-hexane diol, 3,4-octane diol, mixtures thereof or the like where caprylyl glycol is typically the most preferred.

When present in the composition, preservatives are added preferably in an amount 0.001 to 5 wt %, more preferably 0.01 to 3 wt % and most preferably 0.02 to 2 wt %, even most preferably 0.25 to 1.5%.

Preferably, the composition comprises a range of other optional ingredients that include antioxidants, binders, buffering agents, colorants, astringents, fragrance, opacifying agents, conditioners, exfoliating agents, pH adjusters, skin sensates, skin soothing agents, and skin healing agents.

The present invention also relates to a method of delivering anti-aging benefit to skin comprising the step of applying a composition of the invention on to the desired skin surface. The method is preferably non-therapeutic/cosmetic.

The invention will now be illustrated with the help of the following non-limiting examples.

### EXAMPLES

Combination of Arginine with Dicarboxylic Acids or Esters that Demonstrate Synergistic Improvement in Gene Expression of Collagen1:

Experiments were carried out to study gene expression of Collagen1 with actives as shown in Table-1 individually and in combination and the data is 12ummarized therein. The methodology used to measure the gene expression is given below:

WS1 cells (human dermal fibroblasts) were grown and maintained in high glucose DMEM (HIMEDIA cat No AL219A) supplemented with 0.03 mM Calcium chloride, 1 mM sodium pyruvate and 10% FBS (Fetal Bovine Serum) at 37° C. with 5% CO₂. 80% confluent cells were trypsinized and plated in 24 well plate at a density of 50,000 cells/well. The cells were incubated at 37° C. with 5% CO₂ for 24 hours. Post incubation, the cells were treated with arginine (0.005%) and varying concentration of pimelic acid, dimethyl pimelate, suberic acid. Azelaic acid (0.0001, 0.001, 0.005%). The treatment was done using individual ingredients and in respective combinations as given in table 1. 24 hours after treatment the cells were harvested for RNA extraction. RNA was extracted using Qiagen RNA extraction kit (Cat No: 74004) as per the instructions given in the kit. After extraction of RNA, cDNA synthesis was done using iscript cDNA synthesis kit (Biorad Cat no: 1708890). 1 g of RNA was used for cDNA synthesis. cDNA template synthesized was used for gene expression studies.

Quantification of collagen gene expression was carried out using real time PCR. 2.5 ml of cDNA was used for setting up the reaction. The PCR reaction mix had 1 ml each of forward and reverse primer, 5 ml of PCR master mix (Sigma Cat No: KCQS00). Amplification was carried out at 95° C. for 30 seconds as the first step, followed by 40 cycles of qRT-PCR at 95° C. for 5 seconds, and at 60° C. for 20 seconds. mRNA expression was measured in triplicate and was normalized to GAPDH (glyceraldehyde 3-phosphate dehydrogenase) expression levels. The relative quantification was done using delta delta Ct method. The primers used in the study are COL1A1 forward primer 5′-GATTCCCTGGACCTAAAGGTGC-3′, COL1A1 reverse primer 5′-AGCCTCTCCATCTTTGCCAGCA-3′, GAPDH forward primer 5′-TGACTTCAACAGCGACACCCA-3′, reverse primer 5′-CACCCTGTTGCTGTAGCCAAA-3′. The relative quantification of gene expression was done after normalization using house keeping gene (GAPDH) and comparing with untreated control.

The fold change of gene ‘expression obtaine’ from cells treated with’ combination of test actives were compared with the gene expression obtained from cells treated with individual ingredients. The gene expression values of collagen obtained after treatment with individual and combination of test actives are given in the table 1.

TABLE 1

| | | | Weight ratio |
|---|---|---|---|
| | | Gene | (Arginine:C₇ to C₉ |
| | | expression | dicarboxylic acid or |
| Example | Actives (wt %) | (Collagen1) | ester thereof) |
| Control | — | 1.0 | — |
| A | 0.005 Arginine | 1.9 | — |
| B1 | 0.0001 Pimelic acid | 1.1 | — |
| B2 | 0.001 Pimelic acid | 2.8 | — |
| B3 | 0.005 Pimelic acid | 3.2 | — |
| C1 | 0.0001 Dimethyl pimelate | 1.2 | — |
| C2 | 0.001 Dimethyl pimelate | 2.5 | — |
| C3 | 0.005 Dimethyl pimelate | 3.8 | — |
| D1 | 0.0001 Suberic acid | 1.6 | — |
| D2 | 0.001 Suberic acid | 2.9 | — |
| D3 | 0.005 Suberic acid | 1.3 | — |
| E1 | 0.0001 Azeleic acid | 1.4 | — |
| E2 | 0.001 Azeleic acid | 1.6 | — |
| E3 | 0.005 Azeleic acid | 1.9 | — |
| 1 | 0.005 Arginine + 0.0001 Pimelic acid | 6.29 | 50:1 |
| 2 | 0.005 Arginine + 0.001 Pimelic acid | 6.99 | 5:1 |
| 3 | 0.005 Arginine + 0.005 Pimelic acid | 5.96 | 1:1 |
| 4 | 0.005 Arginine + 0.0001 Dimethyl pimelate | 5.56 | 50:1 |
| 5 | 0.005 Arginine + 0.001 Dimethyl pimelate | 5.54 | 5:1 |
| 6 | 0.005 Arginine + 0.005 Dimethyl pimelate | 3.66 | 1:1 |
| 7 | 0.005 Arginine + 0.0001 Suberic acid | 5.62 | 50:1 |
| 8 | 0.005 Arginine + 0.001 Suberic acid | 6.93 | 5:1 |
| 9 | 0.005 Arginine + 0.005 Suberic acid | 4.97 | 1:1 |
| 10 | 0.005 Arginine + 0.0001 Azeleic acid | 4.48 | 50:1 |
| 11 | 0.005 Arginine + 0.001 Azeleic acid | 4.64 | 5:1 |
| 12 | 0.005 Arginine + 0.005 Azeleic acid | 4.14 | 1:1 |

The data in the table-1 above indicates that a combination of arginine with select dicarboxylic acids or esters delivers synergistic improvement in gene expression of collagen1 which is known to be an indicator of anti-aging benefit to skin.

Combination of Lysine with Dicarboxylic Acids or Esters to Demonstrate that it does not Give any Significant Improvement in Gene Expression of Collagen1:

Experiments were carried out to study gene expression of Collagen1 with amino acid lysine and dicarboxylic acids or esters individually and in combination as shown in Table-2 and the data is 14ummarized therein. The methodology used to measure the gene expression is the same as used earlier.

TABLE 2

| | | Gene expression |
|---|---|---|
| Example | Actives (wt %) | (Collagen1) |
| Control | — | 1.0 |
| F | 0.005 Lysine | 0.8 |
| G1 | 0.0001 Pimelic acid | 1.1 |
| G2 | 0.001 Pimelic acid | 2.4 |
| G3 | 0.005 Pimelic acid | 3.0 |
| H1 | 0.0001 Dimethyl pimelate | 1.2 |
| H2 | 0.001 Dimethyl pimelate | 2.4 |
| H3 | 0.005 Dimethyl pimelate | 3.7 |
| J1 | 0.0001 Suberic acid | 1.7 |
| J2 | 0.001 Suberic acid | 2.8 |
| J3 | 0.005 Suberic acid | 1.4 |
| K1 | 0.0001 Azeleic acid | 1.3 |
| K2 | 0.001 Azeleic acid | 1.6 |
| K3 | 0.005 Azeleic acid | 2.0 |
| G4 | 0.005 Lysine + 0.0001 Pimelic acid | 1.5 |
| G5 | 0.005 Lysine + 0.001 Pimelic acid | 1.2 |
| G6 | 0.005 Lysine + 0.005 Pimelic acid | 1.6 |
| H4 | 0.005 Lysine + 0.0001 Dimethyl pimelate | 1.3 |
| H5 | 0.005 Lysine + 0.001 Dimethyl pimelate | 1.1 |
| H6 | 0.005 Lysine + 0.005 Dimethyl pimelate | 1.6 |
| J4 | 0.005 Lysine + 0.0001 Suberic acid | 2.5 |
| J5 | 0.005 Lysine + 0.001 Suberic acid | 1.0 |
| J6 | 0.005 Lysine + 0.005 Suberic acid | 0.9 |
| K4 | 0.005 Lysine + 0.0001 Azeleic acid | 0.8 |
| K5 | 0.005 Lysine + 0.001 Azeleic acid | 1.0 |
| K6 | 0.005 Lysine + 0.005 Azeleic acid | 0.7 |

The data in the table-2 above indicates that combination of lysine with a dicarboxylic acid or an ester does not give any significant anti-aging benefit as compared to a combination comprising arginine.

Additional experiments were carried out as shown in table-3 below and gene expression for collagen 1 was assessed as described earlier.

TABLE 3

| | | | Weight ratio |
|---|---|---|---|
| | | Gene | (Arginine:C7 to C9 |
| | | expression | dicarboxylic acid or |
| Example | Actives (wt %) | (Collagen1) | ester thereof) |
| Control | — | 1.0 | — |
| L1 | Arginine 0.005 | 1.5 | — |
| L2 | Arginine 0.05 | 3.3 | — |
| L3 | Arginine 0.1 | 4.4 | — |
| M1 | Pimelic acid 0.0025 | 3.0 | — |
| M2 | Pimelic acid 0.025 | 3.9 | — |
| M3 | Pimelic acid 0.002 | 2.7 | — |
| N1 | Dimethyl pimelate 0.0025 | 2.2 | — |
| N2 | Dimethyl pimelate0.025 | 3.5 | — |
| N3 | Dimethyl pimelate 0.002 | 2.5 | — |
| O1 | Suberic 0.0025 | 2.7 | — |
| O2 | Suberic acid 0.025 | 3.7 | — |
| O3 | Suberic acid 0.002 | 2.5 | — |
| P1 | Azelaic 0.0025 | 1.7 | — |
| P2 | Azelaic 0.025 | 2.4 | — |
| P3 | Azelaic 0.002 | 1.6 | — |
| 13 | Arginine 0.005 + Pimelic acid 0.0025 | 5.7 | 2:1 |
| 14 | Arginine 0.1 + Pimelic acid 0.025 | 8.5 | 4:1 |
| 15 | Arginine 0.05 + Pimelic acid 0.002 | 6.6 | 25:1 |
| 16 | Arginine 0.005 + Dimethyl pimelate 0.0025 | 4.4 | 2:1 |
| 17 | Arginine 0.1 + Dimethyl pimelate 0.025 | 8.5 | 4:1 |
| 18 | Arginine 0.05 + Dimethyl pimelate 0.002 | 6.4 | 25:1 |
| 19 | Arginine 0.005 + Suberic acid 0.0025 | 5.5 | 2:1 |
| 20 | Arginine 0.1 + Suberic acid 0.025 | 8.2 | 4:1 |
| 21 | Arginine 0.05 + Suberic acid 0.002 | 6.6 | 25:1 |
| 22 | Arginine 0.005 + Azelaic acid 0.0025 | 4.5 | 2:1 |
| 23 | Arginine 0.1 + Azelaic acid 0.025 | 7.3 | 4:1 |
| 24 | Arginine 0.05 + Azelaic acid 0.002 | 5.6 | 25:1 |

It is to be understood that the experiments described above were conducted in an invitro assay to evaluate gene expression behaviour. It is expected that the concentrations to be actually used to prepare a composition for topical use would be vastly different. The concentrations could be orders of magnitude higher due to the following reasons that affect the difference in concentration in the bulk as compared to that at the cellular level. The composition may be formulated as an emulsion or a gel with very many additional ingredients. The concentration of the desired actives in the oil phase and in the water phase, is expected to be very different. They may also have very different physical and hydrodynamic properties like partition coefficients, diffusional rates, convective transport rates, rheological properties etc. Therefore, it is expected that the concentrations to be used when formulated as a composition would be very different, often orders of magnitude higher, from that at the cellular level, which is the concentration at which the experiments were carried out.

1. A personal care composition comprising:
(i) arginine;
(ii) a C₇ to C₉ dicarboxylic acid or ester thereof; and
(iii) a cosmetically acceptable vehicle;
wherein a weight ratio of arginine to the C₇ to C₉ dicarboxylic acid or ester thereof is from 50:1 to 1:1, with the proviso that if the C₇ to C₉ dicarboxylic acid or ester thereof is dimethyl pimelate, the weight ratio of arginine and dimethyl pimelate is not 1:1.

2. The composition as claimed in claim 1, wherein the dicarboxylic acid or ester thereof is selected form one or more of pimelic acid, dimethyl pimelate, suberic acid, and azelaic acid.

3. The composition as claimed in claim 2, wherein the dicarboxylic acid or ester thereof is pimelic acid.

4. The composition as claimed in claim 1, wherein the composition comprises from 0.1 to 5.0 wt % of arginine.

5. The composition as claimed in claim 1, wherein the composition comprises from 0.05 to 1.5 wt % of the dicarboxylic acid or ester thereof.

6. The composition as claimed in claim 1, wherein the weight ratio of arginine to the dicarboxylic acid or ester thereof is from 25:1 to 1:1.

7. The composition as claimed in claim 1, wherein the weight ratio of arginine to the dicarboxylic acid or ester thereof is from 5:1 to 1:1.

8. The composition as claimed in claim 1, wherein the weight ratio of arginine to the dicarboxylic acid or ester thereof is from 4:1 to 1:1.

9. The composition as claimed in claim 1, wherein the weight ratio of arginine to the dicarboxylic acid or ester thereof is from 2:1 to 1:1.

10. A method of delivering anti-aging benefit to skin comprising applying the composition as claimed in claim 1 on to a desired skin surface.

11. The composition as claimed in claim 1 further comprising at least one skin glow agent.

12. The composition as claimed in claim 1 further comprising at least one nonionic surfactant.

13. A personal care composition comprising:
(i) arginine;
(ii) a C₇ to C₉ dicarboxylic acid or ester thereof selected from pimelic acid, dimethyl pimelate, suberic acid, or azelaic acid; and
(iii) a cosmetically acceptable vehicle;
wherein a weight ratio of arginine to the C₇ to C₉ dicarboxylic acid or ester thereof is from 50:1 to 25:1.

14. A personal care composition comprising:
(i) arginine;
(ii) a C₇ to C₉ dicarboxylic acid or ester thereof selected from pimelic acid, dimethyl pimelate, suberic acid, or azelaic acid; and
(iii) a cosmetically acceptable vehicle;
wherein a weight ratio of arginine to the C₇ to C₉ dicarboxylic acid or ester thereof is from 5:1 to 2:1.
